(19) United States
(12) Patent Application Publication
(10) Pub. No.: US 2023/0372560 A1
(43) Pub. Date: Nov. 23, 2023

(54) DEVICE FOR SANITIZING BANKNOTES

(71) Applicant: Roberto SERPILLI, Chiaravalle (IT)

(72) Inventor: Roberto SERPILLI, Chiaravalle (IT)

(21) Appl. No.: 18/029,699

(22) PCT Filed: Sep. 27, 2021
(86) PCT No.: PCT/IB2021/058787

§ 371 Date: Mar. 31, 2023

(30) Foreign Application Priority Data
Oct. 2, 2020 (IT) 202020000005569

(51) Int. Cl. A61L 2/10; A61L 2/26; B65G 15/12

(52) U.S. Cl. CPC A61L 2/10; A61L 2/26; B65G 15/12; A61L 2202/11; A61L 2202/123; A61L 2202/122; A61L 2202/14

(57) ABSTRACT

The present invention relates to a device for sanitizing banknotes and similar items (1) comprising: a container (2) where are defined an inlet (6) for inserting at least one banknote (S) or similar item into the container (2) and an outlet (7) for extracting at least one banknote (S) or similar item from the container (2); a first conveyor system (8) arranged in the container (2) and configured to convey at least one banknote (S) or similar item from the inlet (6) to a predetermined area between the inlet (6) and the outlet (7) picking said at least one banknote (S) or similar item at a first zone of said at least one banknote (S) or similar item; and a second conveyor system (15) arranged in the container (2) and configured to convey said at least one banknote (S) or similar item from said predetermined area to the outlet (7) picking said at least one banknote (S) or similar item at a second zone of said at least one banknote (S) or similar item, wherein said second zone is other than said first zone. Such a device for sanitizing banknotes and similar items (1) also comprises at least one first UVC ray emitter (24), or at least one first ozone lamp, arranged in the container (2) in such a way as to directly irradiate and sanitize, in use, the entire lower face of at least one banknote (S) or similar item conveyed from the inlet (6) to the outlet (7) by the first conveyor system (8) and the second conveyor system (15); and at least one second UVC ray emitter (26), or at least one second ozone lamp, arranged in the container (2) in such a way as to directly irradiate and sanitize, in use, the entire upper face of at least one banknote (S) or similar item conveyed from the inlet (6) to the outlet (7) by the first conveyor system (8) and the second conveyor system (15).

The present invention relates to a device for sanitizing banknotes and similar items.

A particularly deeply felt issue today derives from the need to manipulate banknotes to carry out payments.

Sometimes, in actual fact, cash payment is mandatory, as it is the only payment method allowed; this can happen, for example, when you have to or wish to buy a product or an item in a sales outlet without any devices suitable for receiving payments in other forms than cash, such as the so-called “POS” payment terminals, which allow payments to be made by electronic money, or by credit, debit or prepaid cards.

What has just been said may also apply to self-checkout machines without devices for payment by credit, debit or prepaid cards; in these cases, in order to pay the amount due, it is necessary to insert coins and/or banknotes in special slots provided in the self-checkout machine.

This determines a high risk, for users of physical money (e.g. banknotes and coins), of coming into contact with viruses and/or bacteria and/or spores and/or moulds and/or other pathogens that may be present on the surfaces of the banknotes and/or coins, which could affect the health of the users.

Devices for disinfecting, or sanitizing, banknotes are known, which however have a number of drawbacks and technical limitations mainly linked to the inadequately effective destruction of viruses and bacteria present on banknotes, and also to their structural complexity and, consequently, to their high manufacturing costs.

The object of the present invention is to solve the prior art issues referred to here above.

Another object of the invention is to provide a device for sanitizing banknotes and similar items, which is particularly effective in destroying viruses and/or bacteria and/or spores and/or moulds and/or other pathogens found on the banknotes.

A further object of the invention is to provide a device for sanitizing banknotes and similar items, which does not have a complex structure and which is easy and economical to make.

Therefore, a specific object of the present invention is a device for sanitizing banknotes and similar items as defined in claim 1 below.

Preferred embodiments are defined in the dependent claims.

Characteristics and advantages will become more obvious from the following description of preferred but not exclusive embodiments illustrated, purely by way of non-limiting example, in the accompanying drawings, wherein:

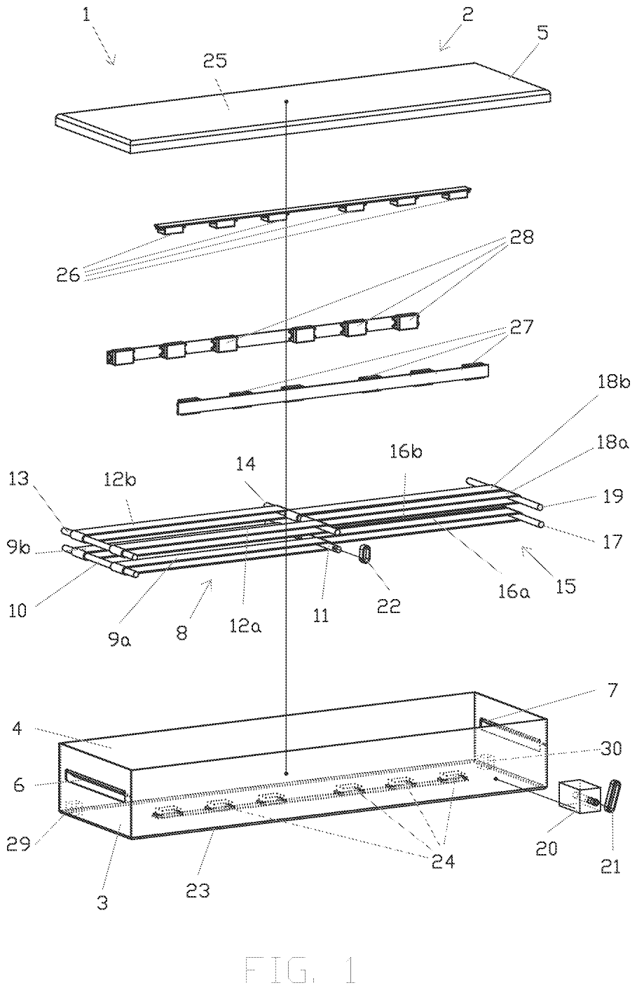

FIG. 1 is an exploded view of a device for sanitizing banknotes and similar items according to the present invention;

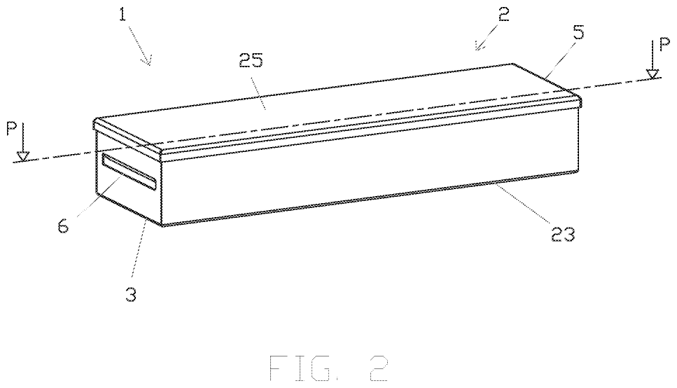

FIG. 2 is an axonometric view of the device shown in FIG. 1, in an assembled configuration;

FIG. 3 is a cross-sectional view along the section line P-P indicated in FIG. 2, in a first phase of operation of said device, wherein a banknote is represented in a first position;

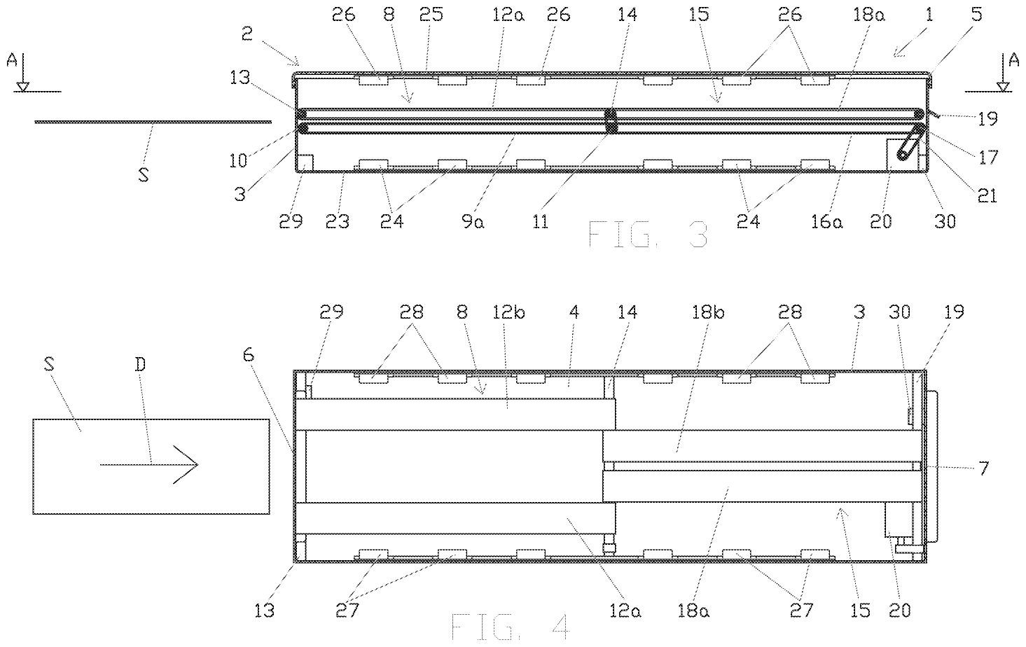

FIG. 4 is a cross-sectional view taken along the section line A-A indicated in FIG. 3;

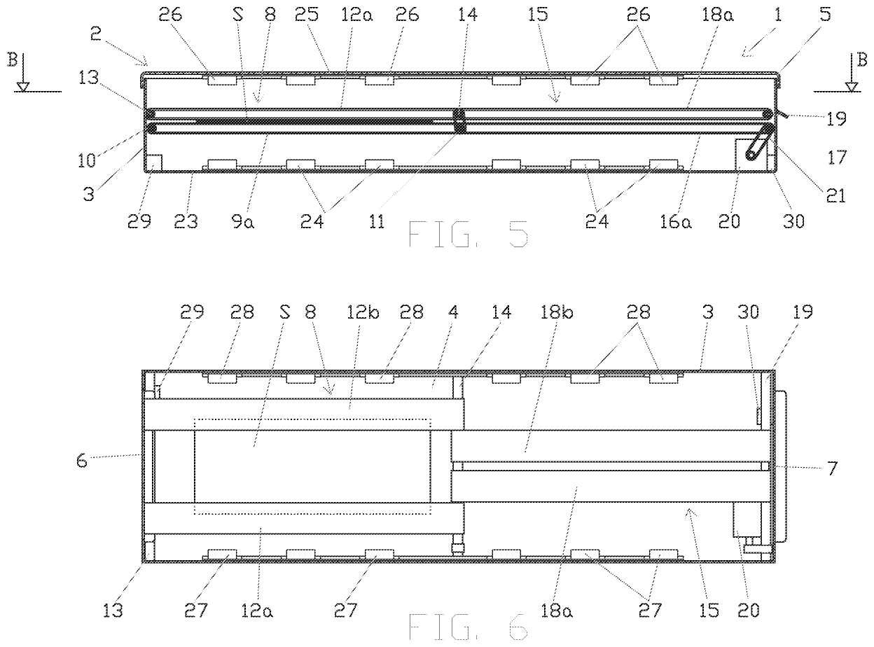

FIG. 5 is a cross-sectional view along the section line P-P indicated in FIG. 2, in a second phase of operation of said device, wherein a banknote is represented in a second position;

FIG. 6 is a cross-sectional view taken along the section line B-B indicated in FIG. 5;

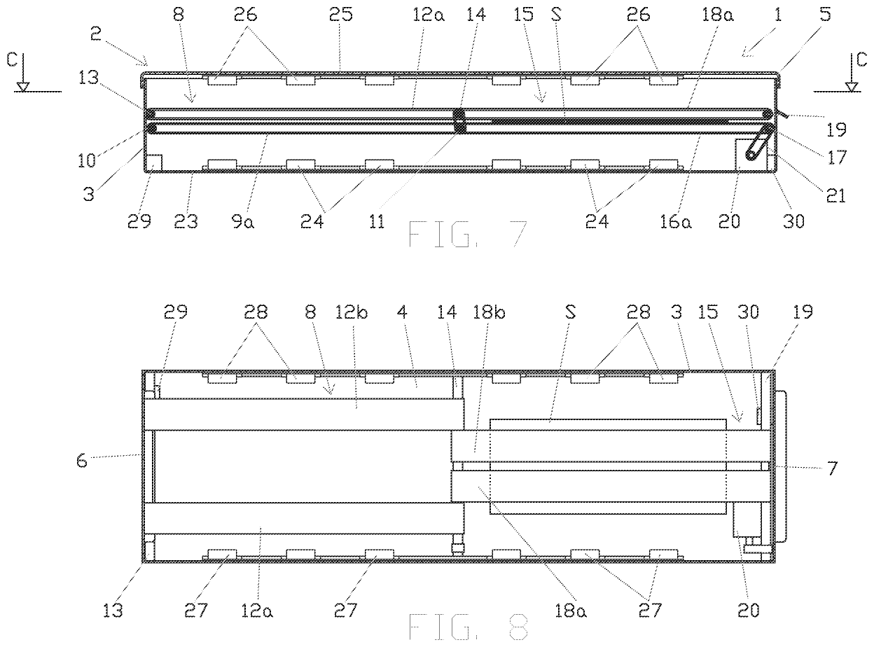

FIG. 7 is a cross-sectional view along the section line P-P indicated in FIG. 2, in a third phase of operation of said device, wherein a banknote is represented in a third position; and

FIG. 8 is a cross-sectional view taken along the section line C-C indicated in FIG. 7.

With reference to the accompanying figures, 1 indicates a device for sanitizing banknotes and similar items according to the present invention.

In particular, this device for sanitizing banknotes and similar items 1 comprises an external casing 2 consisting of a hollow base 3, wherein a compartment 4 is defined, and a lid 5 namely a cover element that can be pressure-coupled to said base 3 in a removable way.

The aforementioned base 3 has, preferably but not limitedly, a substantially parallelepiped shape.

In two opposite faces of the base 3 there are an inlet opening 6 and an outlet opening 7 to allow for the introduction of banknotes S to be treated and the removal of treated banknotes S, respectively.

Inside the base 3 there is a first conveyor system 8 arranged adjacent to the inlet opening 6 and substantially at the same height as the latter.

The first conveyor system 8 comprises a pair of lower conveyor belts 9a, 9b, with synchronous movement, spaced out and parallel to each other and positioned so as to be parallel to the forward feeding direction D of the banknotes S inside the device for sanitizing banknotes and similar items 1.

The conveyor belts of the pair of lower conveyor belts 9a, 9b are wound on a first shaft 10 and a second shaft 11 mounted, parallel to each other, inside the base 3 in a rotatable way around their respective axis and in an orthogonal direction to the aforementioned forward feeding direction D.

The first conveyor system 8 also comprises a pair of upper conveyor belts 12a, 12b, with synchronous movement, in a parallel direction to the forward direction D of the banknotes S and arranged at the same distance existing between the lower conveyor belts 9a, 9b.

The conveyor belts of the above-mentioned pair of upper conveyor belts 12a, 9b are, furthermore, wound on a third shaft 13 and a fourth shaft 14 mounted, parallel to each other, inside the base 3 in a rotatable way around their respective axis and in an orthogonal direction to the forward feeding direction D of the banknotes S inside the device for the sanitizing of banknotes and similar items 1.

This pair of upper conveyor belts 12a, 12b is arranged above the aforementioned pair of lower conveyor belts 9a, 9b at a distance such as to allow the introduction, through the inlet opening 6, and the passage of a banknote S in fully extended configuration, in the space between them.

In particular, the two conveyor belts 12a, 12b of the aforementioned pair of upper conveyor belts 12a, 12b are arranged in such positions as to match, respectively, the underlying conveyor belts 9a, 9b of the pair of lower conveyor belts 9a, 9b.

Downstream of the first conveyor system 8, i.e. between this and the outlet opening 7, a second conveyor system 15 is provided.

The second conveyor system 15 also comprises a pair of lower conveyor belts 16a, 16b, with synchronous movement, parallel to each other and oriented in a parallel direction to the aforementioned forward feeding direction D.

The lower conveyor belts 16a, 16b of the second conveyor system 15 are wound on the aforementioned second shaft 11, in the space between the lower conveyor belts 9a, 9b of the first conveyor system 8, and on a fifth shaft 17 mounted, parallel to the second shaft 11, inside the base 3 in a rotatable manner around its axis and directed orthogonally to the forward feeding direction D of the banknotes S inside the device for sanitizing banknotes and similar items 1.

Specifically, the first shaft 10, the second shaft 11 and the fifth shaft 17 are located inside the base 3 substantially at the same height; this means that the upper surfaces of the pair of lower conveyor belts 9a, 9b of the first conveyor system 8 and of the pair of lower conveyor belts 16a, 16b of the second conveyor system 15 are substantially coplanar.

The second conveyor system 15 also comprises a pair of upper conveyor belts 18a, 18b, with synchronous movement, in a parallel direction to the forward direction D of the banknotes S and arranged at the same distance existing between the respective lower conveyor belts 16a, 16b underlying them.

Furthermore, the upper conveyor belts 18a, 18b of the second conveyor system 15 are wound on the aforementioned fourth shaft 14, in the space between the upper conveyor belts 12a, 12b of the first conveyor system 8, and on a sixth shaft 19 mounted, parallel to the fourth shaft 14, inside the base 3 in a rotatable manner around its axis and directed orthogonally to the aforementioned forward feeding direction D of the banknotes S.

This pair of upper conveyor belts 18a, 18b of the second conveyor system 15 is arranged above the related pair of lower conveyor belts 16a, 16b at a distance such as to allow the passage of a banknote S, in a fully extended configuration, in the space between them and the removal of said banknote S through the outlet opening 7.

In particular, the upper conveyor belts 18a, 18b of the second conveyor system 15 are arranged in such positions as to match the underlying lower conveyor belts 16a, 16b of the same conveyor system 15, respectively.

The third shaft 13, the fourth shaft 14 and the sixth shaft 19 are located inside the base 3 substantially at the same height; this means that the lower surfaces of the pair of upper conveyor belts 12a, 12b of the first conveyor system 8 and of the pair of upper conveyor belts 18a, 18b of the second conveyor system 15 are substantially coplanar.

To drive the first conveyor system 8 and the second conveyor system 15, a motor 20 is provided, arranged in proximity to the outlet opening 7 and connected, by means of a transmission belt 21, to the fifth shaft 17 to allow the rotation of this shaft on its own axis and therefore the sliding, i.e. the forward feeding according to the aforementioned forward feeding direction D, of the pair of lower conveyor belts 9a, 9b of the first conveyor system 8 and of the pair of lower conveyor belts 16a, 16b of the second conveyor system 15.

To transfer the drive from the two pairs of lower conveyor belts 9a, 9b, 16a, 16b to the two pairs of upper conveyor belts 12a, 12b, 18a, 18b, another transmission belt 22 is provided, mounted on the second shaft 11 and on the fourth shaft 14, so as to transmit rotation from said second shaft 11 to said fourth shaft 14 when the former rotates on its respective axis.

Furthermore, on the inner side of the bottom 23 of the base 3 there is a first plurality of emitters of UVC rays 24, i.e. type C ultraviolet rays, arranged in such a way as to substantially cover the entire longitudinal extension of the base 3, i.e. the extension thereof according to the aforementioned forward direction D, and facing the two pairs of lower conveyor belts 9a, 9b, 16a, 16b so as to directly irradiate the lower exposed surfaces of the banknotes S passing through the first conveyor system 8 and the second conveyor system 15, i.e. the lower surfaces of the same banknotes S not covered by said lower conveyor belts 9a, 9b, 16a, 16b.

Similarly, on the inner side of the upper wall 25 of the lid 5 there is a second plurality of emitters of UVC rays 26, i.e. type C ultraviolet rays, arranged in such a way as to substantially cover the entire longitudinal extension of the lid 5, i.e. the extension thereof according to the aforementioned forward direction D, and facing the two pairs of upper conveyor belts 12a, 12b, 18a, 18b when said lid 5 is applied on to the base 3, so as to directly irradiate the upper exposed surfaces of the banknotes S passing through the first conveyor system 8 and the second conveyor system 15, i.e. the upper surfaces of the same banknotes S not covered by said upper conveyor belts 12a, 12b, 18a, 18b.

As can be seen from FIGS. 5 and 6, during conveying of the banknote S by means of the first conveyor system 8 described above, the same banknote S is irradiated, from above and below, at its central strip not covered by the pair of upper conveyor belts 12a, 12b and the pair of lower conveyor belts 9a, 9b.

When, on the other hand, the same banknote S is conveyed by the second conveyor system 15, it is irradiated, from above and below, at two lateral longitudinal strips (externally to the pair of upper conveyor belts 18a, 18b and to the pair of lower conveyor belts 16a, 16b of the second conveyor system 15) and at a central longitudinal strip (comprised between the pair of upper conveyor belts 18a, 18b and the pair of lower conveyor belts 16a, 16b of the second conveyor system 15), i.e. at the strips not covered by the pair of upper conveyor belts 18a, 18b and the pair of lower conveyor belts 16a, 16b of the second conveyor system 15 (see FIGS. 7 and 8).

Wanting to expand on the concept explained above, the first conveyor system 8 and the second conveyor system 15 are configured in such a way as to allow the conveying of banknotes S from the inlet opening 6 to the outlet opening 7 by making contact to, and therefore covering, different longitudinal areas or strips of the banknotes S so that during the passage of each banknote S through the first conveyor system 8 at least one upper longitudinal strip and the underlying at least one lower longitudinal strip of this banknote S are irradiated (see FIGS. 5 and 6) and that during its passage through the second conveyor system 15 all the remaining upper and lower longitudinal strips of the banknote S are irradiated (see FIGS. 7 and 8), so as to obtain total and direct irradiation thereof.

Optionally, a third plurality of UVC ray emitters 27 and a fourth plurality of UVC ray emitters 28 are provided on the inner faces of the two opposite sides of the base 3 not affected by the inlet opening 6 and the outlet opening 7—respectively, both facing the first conveyor system 8 and the second conveyor system 15 and therefore also the side edges of the banknotes S passing through them.

At the inlet opening 6 there is an inlet sensor 29 (for example a presence sensor) suitable for detecting the insertion of the banknotes S through the same inlet opening 6 and operatively connected to the motor 20 and to the aforementioned four pluralities of UVC ray emitters 24, 26, 27, 28, so that when a banknote S is inserted through the inlet opening 6 (see FIGS. 3 and 4), this inlet sensor 29 transmits a signal for automatically controlling the activation of the motor 20 and the switching on of the four pluralities of UVC ray emitters 24, 26, 27, 28 to allow direct and complete irradiation of the banknotes S during their transfer from inlet opening 6 to outlet opening 7.

At the inlet opening 7 an inlet sensor 30 is provided instead (for example a presence sensor) suitable for detecting the banknotes S reaching the outlet opening 7 and also operatively connected to the motor 20 and to the aforementioned four pluralities of UVC ray emitters 24, 26, 27, 28, so that the same outlet sensor 30 transmits a signal for automatically controlling the deactivation of the motor 20 and the switching off of the four pluralities of UVC ray emitters 24, 26, 27, 28 when the banknotes S reach the outlet opening 7.

Optionally, a timer can be provided that is operatively connected to the output sensor 30 and operating in such a way as to control, in any case, the switching off of the four pluralities of UVC ray emitters 24, 26, 27, 28 should the output sensor 30 not detect the presence of a banknote or similar item within a certain period of time from detection by the inlet sensor 29.

Ozone lamps can be provided in place of or in addition to the first plurality of UVC ray emitters 24 and the second plurality of UVC ray emitters 26, and the possible third plurality of UVC ray emitters 27 and fourth plurality of UVC ray emitters 28.

To improve the conveying of the banknotes S through the first conveyor system 8 and the second conveyor system 15, it is possible to provide a pressing system (not shown in the attached figures) acting on the two pairs of lower conveyor belts 9a, 9b, 16a, 16b and/or on the two pairs of upper conveyor belts 12a, 12b, 18a, 18b, to help the adhesion of the same banknotes S to said conveyor belts and therefore their conveying.

According to a variant of the present invention not shown in the accompanying figures, in replacement of the two pairs of upper conveyor belts 12a, 12b, 18a, 18b and of the relative shafts 13, 14, 19 it is possible to provide a plurality of idle rollers with horizontal rotation axis, possibly kept pushing against the underlying lower conveyor belts 9a, 9b, 16a, 16b by means of a thrust system acting downwards.

In this case, these idle rollers operate in such a way as to push the banknotes S towards the underlying lower conveyor belts 9a, 9b, 16a, 16b, ensuring adhesion to them and supporting their forward feeding, made possible thanks to the driving action of the same lower conveyors belts 9a, 9b, 16a, 16b.

According to another variant of the present invention not shown in the accompanying figures, in replacement of the two pairs of lower conveyor belts 9a, 9b, 16a, 16b and of the related shafts 10, 11, 17 it is possible to provide a plurality of idle rollers with horizontal rotation axis, possibly kept pressurised on the overlying lower conveyor belts 12a, 12b, 18a, 18b by means of a thrust system acting from the bottom upwards.

In this case, these idle rollers operate in such a way as to push the banknotes S towards the overlying upper conveyor belts 12a, 12b, 18a, 18b, ensuring adhesion to them and supporting their forward feeding, made possible thanks to the driving action of the same upper conveyors belts 12a, 12b, 18a, 18b.

According to further embodiments of the present invention not shown in the accompanying figures, the two conveyor systems 8, 15 described above might not be coplanar, but rather arranged one below or above the other, provided that during the entire pathway of the banknote S from the inlet 6 to the outlet 7 of the external envelope 2, both the upper surface and the lower surface of the banknote S are entirely and directly irradiated.

As can be inferred from the above description, the device for sanitizing banknotes and similar items 1, according to the present invention, allows complete sanitizing of the banknotes S to be obtained by direct irradiation of all the surfaces of the banknotes.

Furthermore, the structure of the aforementioned device for sanitizing banknotes and similar items 1 is not particularly complex and therefore can be implemented in a simple and economical way.

It is understood that the above has been described by way of non-limiting example, so that any construction variations should be considered as falling within the scope of the present technical solution, as described above and claimed below.

1. Device for sanitizing banknotes (1) comprising:
a container (2) where are defined an inlet (6) for inserting at least one banknote (S) into the container (2) and an outlet (7) for extracting at least one banknote (S) from the container (2);
a first conveyor system (8) arranged in the container (2) and configured to convey at least one banknote (S) according to a forward direction (D) from the inlet (6) to a predetermined area between the inlet (6) and the outlet (7) picking said at least one banknote (S) at a first zone of said at least one banknote (S); wherein the first conveyor system (8) comprises at least one first conveyor belt (9a) located adjacent to the inlet (6) and at least one and conveyor belt (12a), or a plurality of revolving elements, arranged above said at least one first conveyor belt (9a) of the first conveyor system (8) such that, in use, said at least one banknote (S) introduced into the inlet (6) is conveyed between said at least one first conveyor belt (9a) and said at least one second conveyor belt (12a), or said plurality of revolving elements, of the first conveyor system (8);
a second conveyor system (15) arranged in the container (2) and configured to convey said at least one banknote (S) from said predetermined area to the outlet (7) picking said at least one banknote (S) at a second zone of said at least one banknote (S), wherein said second zone is other than said first zone; wherein second conveyor system (15) comprises at least one first conveyor belt (16a) arranged between said at least one first conveyor belt (9a) of the first conveyor system (8) and the outlet (7) in a horizontally staggered way, according to a direction transversal to the forward direction (D), with respect to said at least one first conveyor belt (9a) of the first conveyor system (8); wherein the second conveyor system (15) comprises at least one second conveyor belt (18a), or a plurality of revolving elements, arranged above said at least one first conveyor belt (16a) of the second conveyor system (15) such that, in use, said at least one banknote (S) is conveyed between said at least one first conveyor belt (16a) and said at least one second conveyor belt (18a), or said plurality of revolving elements, of the second conveyor system (15);
at least one first UVC ray emitter (24), or at least one first ozone lamp, arranged in the container (2) in such a way as to directly irradiate and sanitize, in use, the entire lower face of at least one banknote (S) conveyed from the inlet (6) to the outlet (7) by the first conveyor system (8) and the second conveyor system (15); and
at least one second UVC ray emitter (26), or at least one second ozone lamp, arranged in the container (2) in such a way as to directly irradiate and sanitize, in use, the entire upper face of at least one banknote (S) conveyed from the inlet (6) to the outlet (7) by the first conveyor system (8) and the second conveyor system (15).

2. Device for sanitizing banknotes (1) according to claim 1, characterised in that it comprises a first sensor (29) configured to detect the introduction of a banknote (S) into the inlet (6) and operatively connected to the first conveyor system (8), to the second conveyor system (15), to said at least one first UVC ray emitter (24), or to said at least one first ozone lamp, and to said at least one second UVC ray emitter (26), or to said at least one second ozone lamp, in such a way that, when a banknote (S) is introduced into the inlet (6), said first sensor (29) emits an electrical signal to order, automatically, the activation of the first conveyor system (8) and of the second conveyor system (15) and the powering on of said at least one first UVC ray emitter (24) or said at least one first ozone lamp, and of said at least one second UVC ray emitter (26) or said at least one second ozone lamp.

3. Device for sanitizing banknotes (1) according to claim 1, characterised in that it comprises a second sensor (30) configured to detect the removal of a banknote (S) from the outlet (7) and operatively connected to the first conveyor system (8), to the second conveyor system (15), to said at least one first UVC ray emitter (24) or to said at least one first ozone lamp, and to said at least one second UVC ray emitter (26) or to said at least one second ozone lamp, in such a way that, when said at least one banknote (S) reaches the outlet (7), said second sensor (30) emits an electrical signal to order, automatically, the deactivation of the first conveyor system (8) and of the second conveyor system (15) and the powering off of said at least one first UVC ray emitter (24) or said at least one first ozone lamp, and of said at least one second UVC ray emitter (26) or said at least one second ozone lamp.

4. (canceled)

5. (canceled)

6. Device for sanitizing banknotes (1) according to claim 1, characterized in that said at least one first conveyor belt (9a) of the first conveyor system (8) and said at least one first conveyor belt (16a) of the second conveyor system (15) are substantially coplanar.

7. (canceled)

8. (canceled)

9. Device for sanitising banknotes (1) according to claim 1, characterised in that it comprises at least one thrusting member or device to push said at least one second conveyor belt (12a), or said plurality of revolving elements, of the first conveyor system (8) against said at least one first conveyor belt (9a) of the first conveyor system (8), or vice versa, and to push said at least one second conveyor belt (18a), or said plurality of revolving items, of the second conveyor system (15) against said at least one first conveyor belt (16a) of the second conveyor system (15), or vice versa.

10. Device for sanitising banknotes (1) according to claim 1, characterised in that the first conveyor system (8) and/or the second conveyor system (15) each comprise first two conveyor belts (9a, 12a; 16a, 18a) arranged above each other and second two conveyor belts (9b, 12b; 16b, 18b) arranged above each other.

11. Device for sanitising banknotes (1) according to claim 1, characterised in that the second conveyor system (15) is arranged, with respect to the first conveyor system (8), at a higher or lower height in such a position that when said at least one banknote (S) is conveyed from the inlet (6) to the outlet (7) by the first conveyor system (8) and the second conveyor system (15), both faces of said at least one banknote (S) are directly and completely irradiated by means of said at least one first UVC ray emitter (24), or at least one first ozone lamp, and said at least one second UVC ray emitter (26), or at least a second ozone lamp.

12. Device for sanitising banknotes (1) according to claim 1, characterised in that it comprises at least one third UVC ray emitter (27), or at least one third ozone lamp, and at least one fourth UVC ray emitter (28), or at least one fourth ozone lamp, arranged in the container (2) opposite each other in such a way as to directly irradiate and sanitize, in use, at least two opposite side edges of said banknote (S) when said banknote (S) is conveyed by the first conveyor system (8) and by the second conveyor system (15).

13. Device for sanitising banknotes (1) according to claim 1, characterised in that it comprises driving means (20) for driving the first conveyor system (8) and the second conveyor system (15).
